SEQUENCE LISTING (6 sequences)

SEQ ID NO: 1 - DNA, 33 bp, Artificial Sequence
  Description of Artificial Sequence  Synthetic Primer
cagagcggccgcatggactataataggatgaac

SEQ ID NO: 2 - DNA, 33 bp, Artificial Sequence
  Description of Artificial Sequence  Synthetic Primer
cccaagcttcagtgcctggaagccccattggtg

SEQ ID NO: 3 - DNA, 26 bp, Artificial Sequence
  Description of Artificial Sequence  Synthetic Primer
aaccctgcggccgctttttttttttg

SEQ ID NO: 4 - DNA, 26 bp, Artificial Sequence
  Description of Artificial Sequence  Synthetic Primer
aaccctgcggccgcttttttttttta

SEQ ID NO: 5 - DNA, 26 bp, Artificial Sequence
  Description of Artificial Sequence  Synthetic Primer
aaccctgcggccgctttttttttttc

SEQ ID NO: 6 - DNA, 12 bp, Artificial Sequence
  Description of Artificial Sequence  Synthetic Primer
cccaagcttggg

(19) United States
(12) Patent Application Publication
(10) Pub. No.: US 2001/0031466 A1
(43) Pub. Date: Oct. 18, 2001

(54) Preparation of sequence libraries from non-denatured RNA and kits therefor

(76) Inventor: Lawrence T. Malek, Brampton (CA)

Correspondence Address: John J. Bruckner, Fulbright & Jaworski L.L.P., Suite 2400, 600 Congress Avenue, Austin, TX, 78701, US

(21) Appl. No.: 09/728,932

(22) Filed: Dec. 1, 2000

Related U.S. Application Data
(60) Provisional application No. 60/168,793, filed on Dec. 2, 1999.

(51) Int. Cl. C12Q 1/68; C12P 19/34

(52) U.S. Cl. 435/6; 435/91.1

(57) ABSTRACT

A method is provided for preparing libraries of DNA sequences from non-denatured RNA. In one embodiment, the method includes: forming a library of target RNA fragments by contacting multiple copies of non-denatured target RNA sequences with a library of random oligonucleotides in the presence of a hydrolytic agent under conditions such that a subgroup of the library of random oligonucleotides hybridizes to the target RNA, whereupon the hydrolytic agent hydrolyzes the target RNA at a site near the 5′ end of each hybridized random oligonucleotide and wherein the 3′ ends of each fragment contains the entire sequence to which a random oligonucleotide in the subgroup hybridized; and forming a library of templates for primer extension.

### RELATED APPLICATIONS

This application claims benefit of priority from U.S. Provisional Patent Application Serial No. 60/168,793, filed Dec. 2, 1999, the entire contents of which are hereby incorporated by reference for all purposes.

### FIELD OF THE INVENTION

The present invention relates to a method for preparing libraries of DNA sequences from non-denatured target RNA.

### BACKGROUND OF THE INVENTION

Roninson et al. (1993) U.S. Pat. No. 5,217,889, discloses methods for isolating and identifying genetic elements that are capable of inhibiting gene function, called genetic suppressor elements. Disclosed examples and methods claims for obtaining genetic suppressor elements and living cells containing genetic suppressor elements both involve randomly fragmented DNA libraries of about 700 base pairs or less.

Shibahara et al. (1987) Nucleic Acids Res. 15:4403-4415, discloses the use of complementary chimeric oligonucleotides containing deoxyribonucleotides and 2′-O-methylribonucleotides for site-specific hydrolysis of an in vitro RNA transcript using RNase H. Chimeric oligonucleotides with 5′-terminal 3 or 4 deoxyribonucleotides directed the RNase H hydrolysis to within one nucleotide of the site on the RNA to which the 5′ nucleotide of the chimeric oligonucleotide hybridized.

Ho et al. (1996) Nucleic Acids Res. 24:1901-1907 and Ho et al.(1998) Nature Biotechnol. 16:59-63, disclose a method for selecting-antisense oligonucleotides by mapping accessible sites on a single known RNA sequence using random sequence libraries of chimeric oligonucleotides and RNase H. The method gave partial hydrolysis of an in vitro transcribed RNA modeling a specific mRNA at sites three nucleotides from the 5′ ends of the hybridized chimeric oligonucleotides. The antisense accessible sites were determined by measuring the lengths of extension products of primers to known internal sequences on the RNA fragment templates.

There thus remains a need to provide methods and kits which enable the preparation of libraries from non-denatured RNA.

The present invention seeks to meet these and other needs.

The present description refers to a number of documents, the content of which is herein incorporated by reference in their entirety.

### SUMMARY OF THE INVENTION

Methods and kits are provided for preparing libraries of DNA sequences from non-denatured target RNA.

According to an embodiment of a method according to the present invention, a library of DNA sequences is prepared from multiple copies of non-denatured target RNA sequences, the method comprising: forming a library of target RNA fragments from multiple copies of non-denatured target RNA sequences; forming a library of templates for primer extension from the library of target RNA fragments; and forming a library of DNA sequences that are complementary to the target RNA fragments from the library of templates for primer extension.

According to another embodiment of a method according to the present invention, a library for the transcription of RNA sequences is prepared from multiple copies of non-denatured target RNA sequences, the method comprising: forming a library of target RNA fragments from multiple copies of non-denatured target RNA sequences; forming a library of templates for primer extension from the library of target RNA fragments; forming a library of DNA sequences that are complementary to the target RNA fragments from the library of templates for primer extension; forming a library of duplex DNA sequences from the library of DNA sequences that are complementary to the target RNA fragments; and forming a library for the transcription of RNA sequences that are complementary to the target RNA fragments from the library of duplex DNA sequences.

According to another embodiment of a method according to the present invention, RNA sequences having antisense activity are identified from multiple copies of non-denatured target RNA sequences, the method comprising: forming a library of target RNA fragments from multiple copies of non-denatured target RNA sequences; forming a library of templates for primer extension from the library of target RNA fragments; forming a library of DNA sequences that are complementary to the target RNA fragments from the library of templates for primer extension; forming a library of duplex DNA sequences from the library of DNA sequences that are complementary to the target RNA fragments; forming a library for the transcription of RNA sequences that are complementary to the target RNA fragments from the library of duplex DNA sequences; introducing the library for the transcription of RNA sequences that are complementary to the target RNA fragments into living cells; selecting those cells which exhibit altered expression of target RNA sequences; and identifying the transcribed RNA sequences from the selected cells.

According to any of the above embodiments, the library of target RNA fragments may be formed by contacting multiple copies of non-denatured target RNA sequences with a library of random oligonucleotides in the presence of a hydrolytic agent, under conditions where a subgroup of the library of random oligonucleotides hybridize to the target RNA, whereupon the hydrolytic agent hydrolyzes the target RNA at a site near the 5′ end of each hybridized random oligonucleotide, and wherein the 3′ ends of each fragment contains the entire sequence to which a random oligonucleotide in the subgroup hybridized.

According to any of the above embodiments, the library of templates for primer extension may be formed by attaching a nucleic acid primer complement sequence to the 3′ end of each target RNA fragment.

According to any of the above embodiments, the library of DNA sequences that are complementary to the target RNA fragments may be formed by extending a nucleic acid primer that is capable of hybridizing to the nucleic acid primer complement sequence using each target RNA fragment as a template for primer extension.

According to any of the above embodiments, the library of duplex DNA sequences may be formed by primer extension using the library of DNA sequences.

According to any of the above embodiments, the library for the transcription of RNA sequences that are complementary to the target RNA fragments may be formed by attaching a duplex promoter sequence to the library of duplex DNA sequences.

In order to provide a clear and consistent understanding of terms used in the present description, a number of definitions are provided hereinbelow.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, N.Y.).

The present description refers to a number of routinely used recombinant DNA (rDNA) technology terms. Nevertheless, definitions of selected examples of such DNA terms are provided for clarity and consistency.

As used herein, “nucleic acid molecule”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense]).

The term “recombinant DNA” as known in the art refers to a DNA molecule resulting from the joining of DNA segments. This is often referred to as genetic engineering. The same is true for “recombinant nucleic acid”.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (e.g. DNA, RNA or hybrids thereof) for practicing the present invention may be obtained according to well known methods.

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 molecules, and they may be adapted to be especially suited to a chosen nucleic acid amplification system. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “DNA” molecule or sequence (as well as sometimes the term “oligonucleotide”) refers to a molecule comprised of the deoxyribonucleotides adenine (A), guanine (G), thymine (T) and/or cytosine (C), often in a double-stranded form, and comprises or includes a “regulatory element” according to the present invention, as the term is defined herein. The term “oligonucleotide” or “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction. Of course and as well known in the art, DNA molecules can also be found in single-stranded form.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5×SSC or 5×SSPE), 5×Denhardt's solution, 1% SDS, and 100 μg/ml denatured carrier DNA (e.g. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al.,1989, supra).

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988, Ann. Reports Med. Chem. 23:295 and Moran et al., 1987, Nucleic Acids Res., 14:5019. Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Although less preferred, labeled proteins could also be used to detect a particular nucleic acid sequence to which it binds. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods (Sambrook et al., 1989, supra). Non-limiting examples of labels include ³H,¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well known methods.

As used herein, a “primer” defines an oligonucleotide which is capable of annealing to a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. Patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise to a specific polypeptide or protein. It will be readily recognized by the person of ordinary skill, that the nucleic acid sequence of the present invention can be incorporated into anyone of numerous established kit formats which are well known in the art.

A “heterologous” (e.g. a heterologous gene) region of a DNA molecule is a subsegment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e.g. SDS gel electrophoresis, gel filtration, centrifugation, ion exchange chromatography . . . ). In addition, the protein of interest can be purified via affinity chromatography using polyclonal or monoclonal antibodies. The purified protein can be used for therapeutic applications.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain −10 and −35 consensus sequences, which serve to initiate transcription and the transcript products contain Shine-Dalgarno sequences, which serve as ribosome binding sequences during translation initiation.

Thus, the term “variant” refers herein to a protein or nucleic acid molecule which is substantially similar in structure and biological activity to the protein or nucleic acid of the present invention.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in most other cellular components.

The present invention also provides antisense nucleic acid molecules which can be used for example to decrease or abrogate the expression of the nucleic acid sequences or proteins of the present invention. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845 and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art.

The present invention relates to a kit for preparing libraries of nucleic acid sequences from non-denatured RNA. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the non-denatured RNA, a container which contains the primers used in the assay, containers which contain enzymes, and containers which contain wash reagents, and containers which contain the reagents.

Having thus generally described the invention, other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

The present invention relates to methods and kits for preparing libraries of DNA sequences from portions of non-denatured RNA. These libraries of DNA sequences may have a variety of utilities including the generation of antisense probes.

According to an embodiment of a method according to the present invention, a library of DNA sequences is prepared from multiple copies of non-denatured target RNA sequences. As used herein, non-denatured target RNA refers to that which has not been subjected to alteration of its native structure through artificial processes; multiple copies refers to RNA mixtures of the same sequence or of different sequences; and library of DNA sequences refers to a mixture of complementary DNA sequences derived from within the same target RNA molecule or from different target RNA molecules in the mixture.

According to the method, multiple copies of non-denatured target RNA sequences may be contacted with a library of random oligonucleotides in the presence of a hydrolytic agent under conditions where a subgroup of the library of random oligonucleotides hybridize to the target RNA, whereupon the hydrolytic agent hydrolyzes the target RNA at a site near the 5′ end of each hybridized random oligonucleotide to form a library of target RNA fragments, wherein the 3′ ends of each fragment contain the entire sequence to which a random oligonucleotide in the subgroup hybridized. A nucleic acid primer complement sequence may then be attached to the 3′ end of each target RNA fragment to form a library of templates for primer extension. A nucleic acid primer that is capable of hybridizing to the nucleic acid primer complement sequence may then be extended, using each target RNA fragment as a template for primer extension, to form a library of DNA sequences that are complementary to the target RNA fragments.

As used herein, a library of random oligonucleotides refers to a mixture of oligonucleotides having been synthesized by the incorporation of more than one nucleotide at each position of their sequence, and hydrolytic agent refers to a chemical entity or enzyme capable of breaking an RNA polymer; for example, by hydrolyzing a phosphodiester bond. According to one embodiment of the method, the random oligonucleotides comprise deoxyribonucleotides, with preferably four 5′-terminal deoxyribonucleotides, and the hydrolytic agent is preferably RNase H. The random oligonucleotides are preferably chimeric, that is having nucleotides other than deoxyribonucleotides at certain positions in their sequence, in order to limit the action of RNase H on the target RNA. The random oligonucleotides may have a defined nucleotide at certain positions in their sequence to direct the hybridization to particular sequences on the target RNA; for example an initiation codon or a ribozyme processing site. The hydrolytic agent hydrolyzes the target RNA at a site near the 5′ end of each hybridized random oligonucleotide, that is preferably within one nucleotide of the nucleotide on the target RNA to which the 5′-terminal nucleotide of the random oligonucleotide hybridizes, in order to form RNA fragments having 3′-terminal hybridization sites for the subgroup of random oligonucleotides that hybridized and directed their formation.

The method may further include taking a nucleic acid primer complement sequence and attaching it to the 3′ end of each target RNA fragment to form a library of templates for primer extension. In one embodiment, a polymer oroligomer of adenosine nucleotides is attached to the 3′ end of each target RNA fragment by extension using a polyadenylate polymerase. In another embodiment, an oligoribonucleotide is attached to the 3′ end of each target RNA fragment by ligation using an RNA ligase.

The method may further include taking a nucleic acid primer that is capable of hybridizing to the nucleic acid primer complement sequence and extending it, using each target RNA fragment as a template for primer extension, to form a library of DNA sequences that are complementary to the target RNA fragments. The nucleic acid primer is preferably composed of deoxyribonucleotides and may contain useful sequences that are not necessarily complementary to the nucleic acid primer complement sequence; for example, 5′-terminal sequences for recognition by restriction endonucleases or RNA polymerases. The nucleic acid primer may contain 3′-terminal sequences that are not complementary to the nucleic acid primer complement sequence but may be complementary to the 3′ ends of the target RNA fragment portion of the template. These 3′-terminal sequences may be used in forming a library of DNA sequences that are complementary to a subset of the target RNA fragments, for example.

The library of DNA sequences generated as described above should be capable of binding to the non-denatured target RNA sequences.

According to another embodiment of a method according to the present invention, a library for the transcription of RNA sequences is prepared from multiple copies of non-denatured target RNA sequences. As used herein, a library for the transcription of RNA sequences refers to a mixture of nucleic acid molecules capable of producing different complementary RNA sequences from within the same target RNA molecule or throughout a mixture of target RNA molecules.

In order to form a library for the transcription of RNA sequences, multiple copies of non-denatured target RNA sequences may be contacted with a library of random oligonucleotides in the presence of a hydrolytic agent under conditions where a subgroup of the library of random oligonucleotides hybridize to the target RNA, whereupon the hydrolytic agent hydrolyzes the target RNA at a site near the 5′ end of each hybridized random oligonucleotide to form a library of target RNA fragments, wherein the 3′ ends of each fragment contains the entire sequence to which a random oligonucleotide in the subgroup hybridized. A nucleic acid primer complement sequence may then be attached to the 3′ end of each target RNA fragment to form a library of templates for primer extension. A nucleic acid primer that is capable of hybridizing to the nucleic acid primer complement sequence may then be extended, using each target RNA fragment as a template for primer extension, to form a library of DNA sequences that are complementary to the target RNA fragments. A library of duplex DNA sequences may then be formed by primer extension using the library of DNA sequences as templates. The library of duplex DNA sequences may then be attached to a duplex promoter sequence to form a library for the transcription of RNA sequences that are complementary to the target RNA fragments.

The method may further include forming a library of duplex DNA sequences by primer extension using the library of DNA sequences as templates. The primer used in forming the library of duplex DNA sequences may be formed from the hybridized target RNA fragments by partial hydrolysis with RNase H, for example. Alternatively, a duplexing primer complement sequence may be attached to the 3′ end of each DNA sequence to form a library of templates for duplex formation, and then a duplexing primer that is capable of hybridizing to the duplexing primer complement sequence is then extended using the library of templates for duplex formation to form a library of duplex DNA sequences.

The method may further include taking a duplex promoter sequence and attaching it to the library of duplex DNA sequences to form a library for the transcription of RNA sequences that are complementary to the target RNA fragments. In one embodiment, the nucleic acid primer may include the promoter sequence, which is attached by extension of the library of duplex DNA sequences using the promoter sequence of the nucleic acid primer as a template. In this embodiment, the nucleic acid primer complement sequence may additionally include a promoter sequence. In another embodiment, a duplex DNA containing the promoter sequence may be ligated to the library of duplex DNA sequences in an orientation such that a library for the transcription of RNA sequences that are complementary to the target RNA fragments may be formed.

The transcribed RNA sequences generated as described above should be capable of binding to the non-denatured target RNA sequences.

According to another embodiment of a method according to the present invention, RNA sequences having antisense activity are identified from multiple copies of non-denatured target RNA sequences. As used herein, antisense activity refers to a change in a cellular characteristic in response to introducing a sequence into the cell that is complementary to a cellular mRNA sequence.

In order to identify RNA sequences with antisense activity, multiple copies of non-denatured target RNA sequences may be contacted with a library of random oligonucleotides in the presence of a hydrolytic agent under conditions where a subgroup of the library of random oligonucleotides hybridize to the target RNA, whereupon the hydrolytic agent hydrolyzes the target RNA at a site near the 5′ end of each hybridized random oligonucleotide to form a library of target RNA fragments, wherein the 3′ ends of each fragment contains the entire sequence to which a random oligonucleotide in the subgroup hybridized. A nucleic acid primer complement sequence may then be attached to the 3′ end of each target RNA fragment to form a library of templates for primer extension. A nucleic acid primer that is capable of hybridizing to the nucleic acid primer complement sequence may then be extended, using each target RNA fragment as a template for primer extension, to form a library of DNA sequences that are complementary to the target RNA fragments. A library of duplex DNA sequences may then be formed by primer extension using the library of DNA sequences as templates. The library of duplex DNA sequences may then be attached to a duplex promoter sequence to form a library for the transcription of RNA sequences that are complementary to the target RNA fragments. The library for the transcription of RNA sequences may then be introduced into living cells. Those cells which exhibit altered expression of target RNA sequences may then be selected. The transcribed RNA sequences from the selected cells may then be identified.

A method is also provided for generating RNA fragments from a mixture of non-denatured target RNA molecules containing many potentially unknown sequences. These target RNA fragments may be useful in preparing libraries for the transcription of RNA sequences that are complementary to the target RNA. Since these libraries have been derived from sequences that are accessible to hybridization on the non-denatured target RNA, they should transcribe RNA with enhanced capabilities of binding to the mRNA, or precursors thereof, when introduced into a living cell. Such libraries, being formed from specifically hydrolyzed non-denatured RNA, should be more effective as antisense RNA expression libraries than the libraries disclosed by Roninson et al. (1993), having been formed as they were from randomly fragmented DNA.

A method is also provided for forming a library of duplex DNA from target RNA fragments that are generated from a mixture of non-denatured target RNA molecules. This library may be used in the sequencing of cloned duplex DNA fragments. The derived sequences may then be used in the selection of antisense oligonucleotides directed against any RNA molecule in the mixture of target RNA, whether its sequence is known or unknown. This method for comprehensively selecting antisense oligonucleotides against an uncharacterized RNA mixture should be more effective than the method disclosed by Hoet al. (1996) for selecting antisense oligonucleotides by mapping accessible sites on a single known RNA sequence.

The present invention also relates to various kits that may be formed in order to perform the various methods of the present invention. Examples of kits include combinations of two or more reagents used in the methods. Specific examples of kits may include a set of random oligonucleotides and ribonuclease H. A kit may further include polyadenylate polymerase. A kit may also further include RNA ligase, and an oligoribonucleotide having the primer complement sequence.

It will be apparent to those skilled in the art that various modifications and variations can be made in the apparatus and methods of the present invention without departing from the spirit or scope on the invention. Thus, it is intended that the present invention cover the modifications and variations of this invention provided they come within the scope of the appended claims and their equivalents. Additionally, the following examples are appended for the purpose of illustrating the claimed invention, and should not be construed so as to limit the scope of the claimed invention.

### EXAMPLE 1

Preparation of HoxB1 RNA model

NT-2 cells (Stratagene) were grown and treated with retinoic acid as described (Pleasure et al. (1992) J. Neurosci. 12, 1802-1815). After 72 h the retinoic acid-treated NT2 cells were harvested and mRNA was prepared using standard methods. Single-stranded cDNA was prepared from the mRNA using an oligo(dT) primer and Thermoscript Reverse Transcriptase (Life Technologies) according to the supplier. Double-stranded cDNA encoding HoxB1 was amplified from the cDNA by PCR using primers specific for the HoxB1 sequence. Two PCR primers, OGS198 (5′ CAG PGC GGC CGC ATG GAC TAT MT AGG ATG MC 3′, SEQ. ID. NO. 1) and OGS200 (5′ CCC MG CTTCAG TGC CTG GM GCC CCA TTG GTG 3′, SEQ. ID. NO. 2), were used to amplify a 945-bp fragment of the HoxB1 cDNA sequence (from 4 to 948 of Accession No. NM_{—}002144.1) and create cloning sites for Not I and Hind II, as underlined. PCR was performed using standard methods. The PCR product was digested with Not I and Hind III restriction endonucleases according to the supplier (New England Biolabs) and ligated into the Not I and Hind III sites of pcDNA3.1(−) (Invitrogen). The resulting plasmid, pcDNA3.1(−)HoxB1, was then used to prepare the HoxB1 RNA model by in vitro transcription.

Plasmid DNA from pcDNA3.1(−)HoxB1 was linearized by digestion with Hind III prior to transcription. A standard transcription reaction contained 50 mM Tris (pH 8.5), 50 mM KCl, 8 mM MgCl₂, 1.5 mM ATP, 1.5 mM GTP, 1.5 mM, CTP, 1.5 mM UTP, 10 mM dithiothreitol, 500 ng plasmid DNA, 25 units ribonuclease inhibitor (Pharmacia) and 60 units T7 RNA polymerase (Pharmacia), in a final volume of 25 μL. The transcription reaction was incubated at 37° C. for 60 min. Thereafter, 1 unit RQ DNase I (Promega) was added and incubation at 37° C. was continued for another 15 min. The reaction mixture was desalted using Bio-gel P6 (BioRad) and the amount of HoxB1 RNA transcript was quantified at A260ₙₘ.

### EXAMPLE 2

Synthesis of the random oligonucleotides libraries RAS5 and RAS6

Two libraries of random oligonucleotides, RAS5 and RAS6, were designed to hybridize at various undetermined sites on the non-denatured target RNA and to direct the action of the hydrolytic agent, ribonuclease H. Thus a library of target RNA fragments would be formed by using ribonuclease H to hydrolyze the target RNA at sites near the 5′ ends of each hybridized random oligonucleotide.

RAS5 and RAS6 were custom synthesized (Keystone Laboratories) in the following manner. DNA synthesis columns containing deoxyinosine 3′-coupled to a control pore glass support (dl-CPG) was used for chemical coupling steps using an equal-molar mixture of all four (A, C, G and U)5′-dimethoxytrityl (DMT)-2′-OMe-ribonucleoside-3′-phosphoramidites. The syntheses of RAS5 and RAS6 involved five and six chemical coupling steps, respectively. The syntheses of RAS5 and RAS6 was then completed by four steps of chemical coupling using an equal-molar mixture of a four(A, C, G and T) 5′-DMT-2′-deoxyribonucleoside-3′-phosphoramidites.

The resulting libraries of random oligonucleotides, RAS5 and RAS6, can be represented by sequences of 10 and 11 nucleotides joined 5′ to 3′ byphosphodiester bonds as follows: 5′-(dN)₄(rNm)₅dl-3′ (RAS5) and 5′-(dN)₄(rNm)₆dl-3′ (RAS6), where 5′-(dN)₄ represents a 5′-terminal tetradeoxyribonucleotide with a mixture of all four (dA, dC, dG and dT) 2′-deoxyribonucleotides at each position, (rNm)₅ and (rNm)₆ represents a pentaribonucleotide and a hexaribonucleotide with a mixture of all four (Am, Cm, Gm and Um) 2′-O-methylated ribonucleotides at each position, and 3′-dl represents a 3′-terminal deoxyinosine.

### EXAMPLE 3

Preparation of a library of target RNA fragments from HoxB1 RNA

A library of target RNA fragments was prepared from HoxB1 RNA, a non-denatured target RNA. Conditions for RNA fragmentation were determined by testing various concentrations of RAS6, a library of random oligonucleotides, and units of RNase H, a hydrolytic agent, in a series of reactions each containing a fixed amount of the HoxB1 RNA model, which was prepared according to Example 1. Each RNA fragmentation reaction contained 20 mM Tris—HCl (pH 8.0), 100 mM KCl, 10 mM MgCl₂, 1 mM dithiothreitol, and 0.1 pmol HoxB1 RNA, in a final volume of 10 μL. In various combinations, 0.1 nmol 1 nmol, or 5 nmol of RAS6 and 0.1 unit, 1 unit or 10 units of ribonuclease H (USB) were added to each reaction. In addition various combinations of no RAS6 and no ribonuclease H control reactions were prepared. All reactions were incubated at 37° C. for 2 min. The reactions were extracted with phenol/chloroform, desalted on G-50 spin columns, and lyophilized to dryness.

OGS200 primer (200 pmol) was labeled in a 50 μL-reaction containing 50 mM Tris—HCl (pH 7.6), 10 mM MgCl₂, 10 mM 2-mercaptoethanol, 200 μCi [y-³²P] ATP (3000 Ci/mmol) (New England Nuclear) and 40 units T4 polynucleotide kinase (USB). The reaction was incubated at 37° C. for 60 min and then stopped by adding 50 μL 20 mM EDTA (pH 8.0). The [5′-³²P] OGS200 primer was purified onBiogel P30, lyophilized to dryness, and dissolved in 40 μL H₂O.

In order to determine the extent of RNA fragmentation under each condition as set forth in this example, the [5′-³²P] OGS200 primer was hybridized to each preparation of RNA fragments and extended using reverse transcriptase to form [5′-³²P] cDNA products. The lengths of [5′-³²P] cDNA products represent the lengths of 3′-terminal fragments of the HoxB1 RNA model. The primer extension was performed in 10 μL-reactions each containing 50 mM Tris—HCl (pH 8.3), 75 mM KCl, 3 mM MgCl₂, 10 mM dithiothreitol, 0.5 mM dATP, 0.5 mM dCTP, 0.5 mMdGTP, 0.5 mM dTTP, 10 pmol [5′-³²P] OGS200 primer, and 100 units M-MLV reverse transcriptase (USB). A 10 μL-aliquot of the primer extension reaction mix was added to each dried preparation of RNA fragments. The reactions were incubated at 37° C. for 60 min and then stopped by adding 2 μL formamide dye. The primer extension products from each preparation of RNA fragments were separated by electrophoresis on a denaturing 6% polyacrylamide gel and the [5′-³²P] cDNA products were detected by autoradiography. The relative RNA fragmentation using various amounts of RAS6 and ribonuclease H are shown in Table 1.

TABLE 1
RNA fragmentation using various amounts of RAS6 and ribonuclease H

| | |
|---|---|
| RAS6 | Ribonuclease H |

| (pmol) | none | 0.1 unit | 1 unit | 10 units |
|---|---|---|---|---|
| 0 | − | − | − | − |
| 100 | − | − | + | ++ |
| 1000 | − | − | ++ | +++ |
| 5000 | − | ++ | +++ | ++++ |

In the absence of RAS6 or ribonuclease H, there was no fragmentation of the HoxB1 RNA as indicated by extension of the [5′-³²P] primer. With 0.1 unit of ribonuclease H, no RNA fragmentation was observed except for at the highest concentration of RAS6. Increasing the amounts of either RAS6 or ribonuclease H increased RNA fragmentation. Reaction conditions with lower amounts of RAS6 could be compensated with higher amounts of ribonuclease H, in order to produce the same degree of RNA fragmentation. For example, similar RNA fragmentation, as indicated by “++” in Table 1, was observed for the following reaction conditions: 5000 pmol with 0.1 unit, 1000 pmol with 1 unit, and 100 pmol with 10 units, of RAS6 and ribonuclease H, respectively. Extending the reaction time from 2 min to 5 min and 10 min resulted in more RNA fragmentation. The highest amounts of RAS6 (5 nmol) and ribonuclease H (10 units) appeared to give the most RNA fragmentation, as indicated by “++++” in Table 1. RNA fragments generated under these reaction conditions gave primer extension products ranging from 100 to 300 nucleotides in length.

### EXAMPLE 4

Preparation of a library of templates for primer extension

A library for primer extension was prepared from HoxB1 RNA fragments by attaching a polymer or oligomer of adenosine nucleotides, a primer complement sequence, to the 3′ end of each target RNA fragment, by using polyadenylate polymerase. Conditions for adding 3′-terminal poly(A) sequences were tested using various preparations of RNA fragments from the HoxB1 RNA model. RNA fragmentation reactions were set up according to Example 3, except that a fixed amount ofribonuclease H (2 units) and various amounts, 1 nmol or 5 nmol, of RAS6 were added to each reaction. Reactions were incubated at 37° C. for 0 min, 2 min or 5 min. The reactions were extracted with phenol/chloroform, desalted on G-50 spin columns, and lyophilized to dryness.

Each polyadenylation reaction contained 20 mM Tris—HCl (pH 7.0), 50 mM KCl, 0.7 mM MnCl₂, 0.2 mM EDTA, 0.25 mM ATP, 10% glycerol, 1 μg acetylated bovine serum albumin and 300 units yeast poly(A) polymerase (USB), in a final volume of 10 μL. A 10 μL-aliquot of the polyadenylation reaction mix was added to each dried preparation of RNA fragments and to a dried aliquot of 100 pmol HoxB1 RNA. The reactions were incubated at 37° C. for 30 min, extracted with phenol/chloroform, desalted on G-50 spin columns, and lyophilized to dryness.

### EXAMPLE 5

Preparation of a library of complementary DNA sequences

A library of complementary DNA sequences was prepared from the 3′-polyadenylated HoxB1 RNA fragments by hybridizing a primer, oligo(dT), to the primer complement sequence, poly(A), and extending the primer using each RNA fragment as a template. Conditions for synthesizing the library of complementary DNA sequences were tested using the 3′-polyadenylated HoxB1 RNA fragments prepared according to Example 4.

The primer was an equal-molar mixture of three oligonucleotides OGS122 (5′ MC CCT GCG GCC GCT TTT TTT TTT TG 3′, SEQ. ID. NO. 3), OGS123 (5′ AAC CCT GCG GCC GCT TTT TTT TTT TA 3′, SEQ. ID. NO. 4), and OGS124 (5′ AAC CCTGCG GCC GCT TTT TTT TTT TC 3′, SEQ. ID. NO. 5). The primer mixture (50 pmol) was labeled in a 15 μL-reaction containing 50 mM Tris—HCl (pH 7.6), 10 mM MgCl₂, 10 mM 2-mercaptoethanol, 100 μCi [y-³²P] ATP (3000 Ci/mmol) and 10 units T4 polynucleotide kinase. The reaction was incubated at 37° C. for 60 min and then stopped by adding 50 μL 20 mM EDTA (pH 8.0). The [5′-³²P] primer mixture was purified on Biogel P30, lyophilized to dryness, and dissolved in 10 μl H₂O.

The [5′-³²P] primer mixture was hybridized to each preparation of RNA fragments and extended using reverse transcriptase to form [5′-³²P] cDNA products. The primer extension was performed in 10 μL-reactions each containing 50 mM Tris—HCl (pH 8.3), 75 mM KCl, 3 mM MgCl₂, 10 mM dithiothreitol, 0.5 mM dATP, 0.5 mM dCTP, 0.5 mM dGTP, 0.5 mM dTTP, 5 pmol [5′-³² P] primer mixture, and 100 units M-MLV reverse transcriptase. A 10 μL-aliquot of the primer extension reaction mix was added to each dried preparation of polyadenylated RNA from Example 4 and to a dried aliquot of 100 pmol HoxB1 RNA. The reactions were incubated at 37° C. for 60 min and then stopped by adding 2 μL formamide dye. The primer extension products from each preparation of RNA fragments were separated by electrophoresis on a denaturing 6% polyacrylamide gel and the [5′-³²P] cDNA products were detected by autoradiography.

Synthesis of cDNA products from the [5′-³²P] primer mixture requires polyadenylation of HoxB1 RNA and its fragments to form templates for primer extension. Polyadenylated HoxB1 RNA allowed annealing of the primer mixture and provided a template for the synthesis of full-length cDNA. Conversely, the HoxB1 without polyadenylation did not anneal to the primer mixture resulting in no cDNA synthesis. Primer extension reactions containing polyadenylated RNA fragments gave a range of cDNA products that were all shorter than the full-length cDNA.

### EXAMPLE 6

Preparation of a library for the transcription of RNA sequences

A library of target RNA fragments was prepared from HoxB1 RNA, a non-denatured target RNA. The RNA fragmentation reaction contained 20 mM Tris—HCl (pH 8.0), 100 mM KCl, 10 mM MgCl₂, 1 mM dithiothreitol, 2.4 pmol HoxB1 RNA, 20 nmol RAS6 and 40 units ribonuclease H, in a final volume of 100 μL. The reaction was incubated at 37° C. for 7 min, extracted with phenol/chloroform, desalted on a G-50 spin column, and lyophilized to dryness. The preparation of HoxB1 RNA fragments was dissolved in 45 μL water.

A library for primer extension was prepared from HoxB1 RNA fragments by 3′-terminal polyadenylation of target RNA fragments. The preparation of HoxB1 RNA fragments was polyadenylated in a reaction that contained 20 mM Tris—HCl (pH 7.0), 50 mM KCl, 0.7 mM MnCl₂, 0.2 mM EDTA, 0.25 mM ATP, 10% glycerol, 1 μg acetylated bovine serum albumin and 2400 units yeast poly(A) polymerase, in a final volume of 100 μL. The reaction was incubated at 37° C. for 30 min, extracted with phenol/chloroform, desalted on a G-50 spin column, and lyophilized to dryness. The preparation of 3′-polyadenylated HoxB1 RNA fragments was dissolved in 45 μL water.

A library of complementary DNA sequences was prepared from the 3′-polyadenylated HoxB1 RNA fragments by primer extension using the RNA fragments as template. An equal-molar mixture of OGS122, OGS123, and OGS124 primers (250 pmol) was added to the preparation of polyadenylated RNA fragments in a total volume of 60 μL. The RNA-primer mixture was heated to 70° C. for 10 min, chilled at 0° C., and then added to a primer extension reaction that contained 50 mM Tris—HCl (pH 8.3), 75 mM KCl, 3 mM MgCl₂, 10 mM dithiothreitol, 0.5 mM dATP, 0.5 mM dCTP, 0.5 mM dGTP, 0.5 mM dTTP, and 250 units M-MLV reverse transcriptase, in a final volume of 100 μL. The reaction was incubated at 37° C. for 60 min.

A library of duplex DNA sequences was prepared from the library of complementary DNA sequences by primer extension using the library of complementary DNA sequences as templates. The 100-μL primer extension reaction was added to a 100-μL reaction that contained 60 mM Tris—HCl (pH 8.0), 8 mM MgCl₂, 2 mM dithiothreitol, 0.4 mM dATP, 0.4 mM dCTP, 0.4 mM dGTP, 0.4 mM dTTP, 52 μM NAD⁺, 10 μg bovine serum albumin, 5 units ribonuclease H, 60 units E. coli DNA polymerase I (New England Biolabs) and 20 units E. coli DNA ligase (New England Biolabs). The 200-μL reaction was incubated at 16° C. for 60 min. Thereafter, 20 units T4 DNA polymerase (New England Biolabs) was added and incubation at 16° C. was continued for another 10 min. The reaction was stopped by adding 10 μL 0.5 M EDTA, extracted with phenol/chloroform, and precipitated with ethanol. The preparation of duplex DNA was dissolved in 20 μL water. The duplex DNA products were separated by electrophoresis on a 1% agarose gel and detected byethidium bromide staining. A broad range of DNA products were observed.

A library for the transcription of RNA sequences that are complementary to the target RNA fragments was prepared by attaching a duplex promoter sequence to the library of duplex DNA sequences. First, the duplex DNA was ligated to Hind III linkers in a 30-μL reaction that contained 50 mM Tris—HCl (pH 7.5), 10 mM MgCl₂, 10 mM dithiothreitol, 1 mM ATP, 25 μg/ml bovine serum albumin, 2 μg phosphorylated Hind III linker (5′ p CCC AAG CTT GGG 3′, SEQ. ID. NO. 6), New England Biolabs) and 800 units T4 DNA ligase (New England Biolabs). The ligation reaction was incubated at 16° C. for 16 h, and then heated at 65° C. for 10 min. Next, the ligated duplex DNA was digested with Not I and Hind III restriction endonucleases according to the supplier and ligated into the Not I and Hind III sites of pLNCX2 (Clontech). The Hind III and Not I sites are indicated by the underlined sequences in the Hind II linkerand OGS122, OGS123, and OGS124. Finally, the ligated DNA in the pLNCX2 retroviral shuttle vector was used for transforming XL10-gold ultracompetent E. coli cells (Stratagene) and ampicilin resistant colonies were selected. The resulting library could be transfected into mammalian cells for transcription from the cytomegalovirus immediate early promoter of antisense HoxB1 RNA sequences, that is, RNA sequences that are complementary to HoxB1 mRNA, the non-denatured target RNA.

Although the present invention has been described hereinabove by way of preferred embodiments thereof, it can be modified without departing from the spirit and nature of the subject invention as defined in the appended claims.

What is claimed is:

1. A method for forming a library of DNA sequences comprising:
forming a library of target RNA fragments by contacting multiple copies of non-denatured target RNA sequences with a library of random oligonucleotides in the presence of a hydrolytic agent under conditions where a subgroup of the library of random oligonucleotides hybridize to the target RNA, whereupon the hydrolytic agent hydrolyzes the target RNA at a site near the 5′ end of each hybridized random oligonucleotide, and wherein the 3′ ends of each fragment contains the entire sequence to which a random oligonucleotide in the subgroup hybridized;
forming a library of templates for primer extension from the library of target RNA fragments; and
forming a library of DNA sequences that are complementary to the target RNA fragments from the library of templates for primer extension.

2. A method according to claim 1, wherein forming a library of templates for primer extension is by attaching a nucleic acid primer complement sequence to the 3′ end of each target RNA fragment.

3. A method according to claim 1, wherein forming a library of DNA sequences that are complementary to the target RNA fragments is by extending a nucleic acid primer that is capable of hybridizing to the nucleic acid primer complement sequence using each target RNA fragment as a template for primer extension.

4. A method for forming a library for the transcription of RNA sequences comprising:
forming a library of target RNA fragments by contacting multiple copies of non-denatured target RNA sequences with a library of random oligonucleotides in the presence of a hydrolytic agent under conditions where a subgroup of the library of random oligonucleotides hybridize to the target RNA, whereupon the hydrolytic agent hydrolyzes the target RNA at a site near the 5′ end of each hybridized random oligonucleotide, and wherein the 3′ ends of each fragment contains the entire sequence to which a random oligonucleotide in the subgroup hybridized;
forming a library of templates for primer extension from the library of target RNA fragments;
forming a library of DNA sequences that are complementary to the target RNA fragments from the library of templates for primer extension;
forming a library of duplex DNA sequences from the library of DNA sequences that are complementary to the target RNA fragments; and
forming a library for the transcription of RNA sequences that are complementary to the target RNA fragments from the library of duplex DNA sequences.

5. A method according to claim 4, wherein forming a library of templates for primer extension is by attaching a nucleic acid primer complement sequence to the 3′ end of each target RNA fragment.

6. A method according to claim 4, wherein forming a library of DNA sequences that are complementary to the target RNA fragments is by extending a nucleic acid primer that is capable of hybridizing to the nucleic acid primer complement sequence using each target RNA fragment as a template for primer extension.

7. A method according to claim 4, wherein forming a library of duplex DNA sequences is by primer extension using the library of DNA sequences as templates.

8. A method according to claim 4, wherein forming a library for the transcription of RNA sequences that are complementary to the target RNA fragments is by attaching a duplex promoter sequence to the library of duplex DNA sequences.

9. A method for identifying RNA sequences having antisense activity comprising:
forming a library of target RNA fragments by contacting multiple copies of non-denatured target RNA sequences with a library of random oligonucleotides in the presence of a hydrolytic agent under conditions where a subgroup of the library of random oligonucleotides hybridize to the target RNA, whereupon the hydrolytic agent hydrolyzes the target RNA at a site near the 5′ end of each hybridized random oligonucleotide, and wherein the 3′ ends of each fragment contains the entire sequence to which a random oligonucleotide in the subgroup hybridized;
forming a library of templates for primer extension from the library of target RNA fragments;
forming a library of DNA sequences that are complementary to the target RNA fragments from the library of templates for primer extension;
forming a library of duplex DNA sequences from the library of DNA sequences that are complementary to the target RNA fragments;
forming a library for the transcription of RNA sequences that are complementary to the target RNA fragments from the library of duplex DNA sequences;
introducing the library for the transcription of RNA sequences that are complementary to the target RNA fragments into living cells;
selecting those cells which exhibit altered expression of target RNA sequences; and
identifying the transcribed RNA sequences from the selected cells.

10. A method according to claim 9, wherein forming a library of templates for primer extension is by attaching a nucleic acid primer complement sequence to the 3′ end of each target RNA fragment.

11. A method according to claim 9, wherein forming a library of DNA sequences that are complementary to the target RNA fragments is by extending a nucleic acid primer that is capable of hybridizing to the nucleic acid primer complement sequence using each target RNA fragment as a template for primer extension.

12. A method according to claim 9, wherein forming a library of duplex DNA sequences is by primer extension using the library of DNA sequences as templates.

13. A method according to claim 9, wherein forming a library for the transcription of RNA sequences that are complementary to the target RNA fragments is by attaching a duplex promoter sequence to the library of duplex DNA sequences.

14. A method for forming a library of DNA sequences comprising:
contacting multiple copies of non-denatured target RNA sequences with a library of random oligonucleotides in the presence of a hydrolytic agent under conditions where a subgroup of the library of random oligonucleotides hybridize to the target RNA, whereupon the hydrolytic agent hydrolyzes the target RNA at a site near the 5′ end of each hybridized random oligonucleotide to form a library of target RNA fragments, wherein the 3′ ends of each fragment contains the entire sequence to which a random oligonucleotide in the subgroup hybridized;
attaching a nucleic acid primer complement sequence to the 3′ end of each target RNA fragment to form a library of templates for primer extension; and
extending a nucleic acid primer that is capable of hybridizing to the nucleic acid primer complement sequence, using each target RNA fragment as a template for primer extension, to form a library of DNA sequences that are complementary to the target RNA fragments.

15. A method according to claim 14, wherein the library of DNA sequences binds to the non-denatured target RNA sequences.

16. A method according to claim 14, wherein the random oligonucleotides comprise deoxyribonucleotides and the target RNA is hydrolyzed by using RNase H.

17. A method according to claim 16, wherein the random oligonucleotides comprise four 5′-terminal deoxyribonucleotides.

18. A method according to claim 14, wherein attaching a nucleic acid primer complement sequence to the 3′ end of each target RNA fragment is performed by extension using a polyadenylate polymerase.

19. A method according to claim 14, wherein attaching a nucleic acid primer complement sequence to the 3′ end of each target RNA fragment is performed by ligation using an RNA ligase.

20. A method according to claim 14, wherein the nucleic acid primer includes a promoter sequence.

21. The method according to claim 20, wherein the nucleic acid primer complement sequence includes a promoter sequence.

22. A method for forming a library for the transcription of RNA sequences comprising:
contacting multiple copies of non-denatured target RNA sequences with a library of random oligonucleotides in the presence of a hydrolytic agent under conditions where a subgroup of the library of random oligonucleotides hybridize to the target RNA, whereupon the hydrolytic agent hydrolyzes the target RNA at a site near the 5′ end of each hybridized random oligonucleotide to form a library of target RNA fragments, wherein the 3′ ends of each fragment contains the entire sequence to which a random oligonucleotide in the subgroup hybridized;
attaching a nucleic acid primer complement sequence to the 3′ end of each target RNA fragment to form a library of templates for primer extension;
extending a nucleic acid primer that is capable of hybridizing to the nucleic acid primer complement sequence, using each target RNA fragment as a template for primer extension, to form a library of DNA sequences that are complementary to the target RNA fragments;
forming a library of duplex DNA sequences by primer extension using the library of DNA sequences as templates; and
attaching a duplex promoter sequence to the library of duplex DNA sequences to form a library for the transcription of RNA sequences that are complementary to the target RNA fragments.

23. The method according to claim 22, wherein the nucleic acid primer includes a promoter sequence, and attaching a duplex promoter sequence is by extension of the DNA duplex, using the promoter sequence of the nucleic acid primer as a template.

24. The method according to claim 23, wherein the nucleic acid primer complement sequence includes a promoter sequence.

25. The method according to claim 22, wherein attaching the duplex promoter sequence to the library of duplex DNA sequences is performed by ligation using a DNA ligase.

26. A method according to claim 22, wherein the library of RNA sequences binds to the non-denatured target RNA sequences.

27. A method according to claim 22, wherein the random oligonucleotides comprise deoxyribonucleotides and the target RNA is hydrolyzed by using RNase H.

28. A method according to claim 27, wherein the random oligonucleotides comprise four 5′-terminal deoxyribonucleotides.

29. A method according to claim 22, wherein attaching a nucleic acid primer complement sequence to the 3′ end of each target RNA fragment is performed by extension using a polyadenylate polymerase.

30. A method according to claim 22, wherein attaching a nucleic acid primer complement sequence to the 3′ end of each target RNA fragment is performed by ligation using an RNA ligase.

31. A method for identifying RNA sequences having antisense activity comprising:
contacting multiple copies of non-denatured target RNA sequences with a library of random oligonucleotides in the presence of a hydrolytic agent under conditions where a subgroup of the library of random oligonucleotides hybridize to the target RNA, whereupon the hydrolytic agent hydrolyzes the target RNA at a site near the 5′ end of each hybridized random oligonucleotide to form a library of target RNA fragments, wherein the 3′ ends of each fragment contains the entire sequence to which a random oligonucleotide in the subgroup hybridized;
attaching a nucleic acid primer complement sequence to the 3′ end of each target RNA fragment to form a library of templates for primer extension;
extending a nucleic acid primer that is capable of hybridizing to the nucleic acid primer complement sequence using each target RNA fragment as a template for primer extension to form a library of DNA sequences that are complementary to the target RNA fragments;
forming a library of duplex DNA sequences by primer extension using the library of DNA sequences as templates;
attaching a duplex promoter sequence to the library of duplex DNA sequences to form a library for the transcription of RNA sequences that are complementary to the target RNA fragments;
introducing the library for the transcription of RNA sequences that are complementary to the target RNA fragments into living cells;
selecting those cells which exhibit altered expression of target RNA sequences; and
identifying the transcribed RNA sequences from the selected cells.
